(19) United States
(12) Patent Application Publication
(10) Pub. No.: US 2016/0150994 A1
(43) Pub. Date: Jun. 2, 2016

(54) Methods and Apparatuses for Characterisation of Body Tissue

(71) Applicant: TI2 Medical PTY LTD, Rhodes, New South Wales (AU)

(72) Inventor: Warren SMITH, New South Wales (AU)

(21) Appl. No.: 14/899,395

(22) PCT Filed: Jun. 19, 2014
(86) PCT No.: PCT/AU2014/050082

§ 371 Date: Dec. 17, 2015

(30) Foreign Application Priority Data
Jun. 19, 2013 (AU) 2013902219

(51) Int. Cl. A61B 5/053; A61B 5/00

(52) U.S. Cl. CPC A61B 5/053; A61B 5/746

(57) ABSTRACT

Methods and apparatuses for characterization of body tissue are disclosed in which bioimpedance across a tissue region of interest is determined. A first electrical signal is applied between an inner pair of current electrodes (B,C) and one or more voltages are measured between an outer pair of sensing electrodes (A,D) and/or between one of the outer pair of sensing electrodes (A,D) and one of the inner pair of current electrodes (B,C). In some embodiments, the methods and apparatuses provide for characterization of electrode-to-tissue contact.

### CROSS-REFERENCE TO RELATED APPLICATIONS

The present application claims priority from Australian provisional patent application no. 2013902219 filed on 19 Jun. 2013, the contents of which are incorporated herein by reference in their entirety.

### TECHNICAL FIELD

The present disclosure relates to methods for characterisation of body tissue, including methods of bioimpedance analysis, and apparatus for use in such methods.

### BACKGROUND

Bioimpedance analysis involves the measurement of the response of a living organism to externally applied electrical current. For example, bioimpedance parameters such as resistance, reactance and phase angle can be recorded, for the purposes of determining blood flow and body composition (e.g., water and fat content).

A standard tetrapolar (four-electrode) method for determination of the bioelectrical impedance of a region of interest of a body (ROI) involves application of a controlled current or controlled voltage waveform between two outer current electrodes connected to body tissue on opposite sides of the ROI, and measurement of the voltage across two inner sensing electrodes located on opposite sides of the ROI at positions inside of the outer current electrodes, the outer and inner electrodes being in a quasi-linear form.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

According to a first aspect, the present disclosure provides a method for characterization of body tissue, the method comprising:

applying a first electrical signal between an inner pair of current electrodes (B,C), the inner pair of current electrodes comprising a first current electrode (B) and a second current electrode (C) connected to tissue at a first side and a second side, respectively, of a tissue region of interest;

measuring one or more voltages between a first sensing electrode (A) and a second sensing electrode (D) of an outer pair of sensing electrodes (A,D) and/or between one of the outer pair of sensing electrodes (A,D) and one of the inner pair of current electrodes (B,C), the voltages resulting from the application of the first electrical signal, wherein the first sensing electrode (A) and the second sensing electrode (D) are connected to tissue at the first side and the second side, respectively, of the tissue region of interest, and at positions outside of the inner pair of electrodes (B,C); and

determining, from the one or more voltage measurements, bioimpedance (Z_{ROI}) across the tissue region of interest.

The method according to the present disclosure contrasts with a standard tetrapolar (four-electrode) method described above by virtue, for example, of the positioning of the pairs of current and sensing electrodes. The positioning of the electrode pairs is exchanged, with the current electrodes being the inner electrodes and the sensing electrodes being the outer electrodes. The present disclosure may therefore be considered to provide a “reciprocal” electrode arrangement.

It has been recognised that the standard four-electrode method can have significant limitations. If, for example, the region of interest (ROI) is at a relatively shallow portion of the body, e.g. in the skin and/or subdermal layers/superficial muscle layers, to maximise the current delivered to the ROI using the standard four-electrode method, the outer current electrodes are normally positioned as close as possible to the ROI but not contacting the inner sensing electrodes. This may mean that in some cases, ideal placement of current electrodes adjacent the ROI may not be possible due to the need to site voltage electrodes between the current electrodes and the ROI, but not on the ROI itself. Furthermore, measured voltage across the inner sensing electrodes becomes very sensitive to the distance between each outer current electrode and the respective, closest, inner sensing electrode. This behaviour introduces error in measurement repeatability when, for example, replacing electrodes in longitudinal studies or when comparing a region of interest of one part of the body, e.g. for one limb, with its corresponding region on another part of the body, e.g. the contralateral limb.

On the other hand, if the outer current electrodes are placed remotely from the inner sensing electrodes in the standard method, bioimpedance measurement can reach a plateau level where it becomes insensitive to electrode placement at the remote locations. Nevertheless such placement is associated with major limitations. In contrast to the preceding example, applied current will course through deeper tissue, thereby reducing measurement sensitivity for any ROI that is at a shallow portion of a body. Further, as the distance between outer current electrodes and their respective closest inner sensing electrodes is increased and/or the size of the electrodes is reduced, so too does the impedance in the region between these electrodes. This can introduce measurement errors and reduce measurement accuracy as a consequence of a phenomenon known as “negative sensitivity zones” This counter-intuitive phenomenon results in a decrease in measured impedance for the ROI with increases in the impedance between outer current electrodes and their respective closest inner sensing electrodes, and vice versa.

The reciprocal method according to the present disclosure may be considered counter-intuitive, since the sensing electrodes, which are used to characterise the tissue region of interest (ROI), are necessarily spaced further away from the region of interest. This means that the voltage measured by the sensing electrodes is measured across a region that can extend substantially beyond the region of interest. However, since current is applied between the inner current electrodes only, the voltage measurement between the outer sensing electrodes is dictated by the bioimpedance of the tissue between the inner current electrodes. Accordingly, it has been determined that the reciprocal arrangement can provide an accurate indication of bioimpedance at the region of interest located between the inner current electrodes. Moreover, it has been found that the arrangement allows for characterisation of the quality of electrode-to-tissue contact at the current electrodes.

In this portion of the summary section, letters in brackets, e.g. (A, B, C, D), have been used to aid recognition of each electrode under discussion. To further aid recognition, reference may be made to FIG. 1, where corresponding lettering has been used, and which Figure is discussed in more detail further below. Nevertheless, it is not intended that the electrode arrangements disclosed in this summary section are limited to any arrangements shown in the Figures. The discussions in this summary section can be considered either in conjunction with, or entirely independently of, the Figures.

An apparatus that may be used to carry out the method described above is also disclosed herein. In particular, in accordance with a second aspect, the present disclosure provides apparatus for characterization of body tissue, the apparatus comprising:

an inner pair of current electrodes (B,C), the inner pair of current electrodes comprising a first current electrode (B) and a second current electrode (C) adapted to connect to tissue at a first side and a second side, respectively, of a tissue region of interest;

a signal generator adapted to apply a first electrical signal between the inner pair of current electrodes (B,C);

an outer pair of sensing electrodes (A,D), the outer pair of electrodes comprising a first sensing electrode (A) and a second sensing electrode (D) adapted to be connected to tissue at the first side and the second side, respectively, of the tissue region of interest, and at positions outside of the inner pair of electrodes; and

a monitoring device adapted to measure one or more voltages between the first sensing electrode (A) and the second sensing electrode (D) of the outer pair of sensing electrodes (A,D) and/or between one of the outer pair of sensing electrodes (A,D) and one of the inner pair of current electrodes (B,C), resulting from the application of the first electrical signal, and determine, from the one or more voltage measurements, bioimpedance (Z_{ROI}) across the tissue region of interest.

In accordance with the first and second aspects, in one embodiment, voltage (V(A-D)), is measured between the outer pair of sensing electrodes (A,D) resulting from the application of the first electrical signal and bioimpedance (Z_{ROI}) across the tissue region of interest is determined from the voltage measurement (V(A-D)). (In general, the notation “V(A-D)” is intended to represent the voltage drop along the path between electrodes A and D, and is thus a scalar difference in voltage between these electrodes. Similar understanding is intended by the notation “V(A-B)”, “V(C-D)”, etc.).

Where voltage V(A-D) is measured between the outer pair of sensing electrodes (A,D), during application of the first electrical signal having a waveform or waveform spectrum suitable for electrical bioimpedance measurement (such as a controlled current AC waveform I(t)), the bioimpedance Z_{ROI} may be determined using equation 1:

$${Z}_{ROI} = \frac{V ( A - D )}{I ( t )} \qquad [ 1 ]$$

Through inclusion of phase-sensitive electronics in the apparatus, impedance Z can be resolved into real (resistive) and imaginary (reactive) components.

Characterisation of electrode-to-tissue contact at the first current electrode (B) may be carried out by measuring a voltage (V(A-B)) between the first electrodes (A,B) resulting from the application of the first electrical signal or a further electrical signal applied between the first and second current electrodes (B,C), and optionally determining, from the voltage measurement (V(A-B)), bioimpedance (Z(A-B)) across tissue between the first electrodes (A,B).

Similarly, characterisation of electrode-to-tissue contact at the second current electrode (C) may be carried out by measuring a voltage (V(C-D)) between the second electrodes (C,D)) resulting from the application of the first electrical signal or a further electrical signal applied between the first and second current electrodes (B,C) and optionally determining, from the voltage measurement (V(C-D)), bioimpedance (Z(C-D)) across tissue between the second electrodes (C,D).

The bioimpedance measurements (Z(A-B), Z(C-D)) provide essentially the transverse impedances (Z_{B}, Z_{C}) beneath the first and second current electrode (B,C), respectively. These impedances in turn will typically be dominated by the respective electrode-to-tissue contact impedances. The transverse impedances (Z_{B}, Z_{C}) can be calculated using equations 2 and 3.

$$\begin{matrix}{Z}_{B} = \frac{V ( A - B )}{I ( t )} & [ 2 ] \\ {Z}_{C} = \frac{V ( C - D )}{I ( t )} & [ 3 ]\end{matrix}$$

The greater the voltage or bioimpedance measurements (V(A-B), Z(A-B), V(C-D), Z(C-D)), the greater the transverse impedances Z_{B}, Z_{C} at the current electrodes (B,C). Higher transverse impedances can indicate that there is poor electrode contact. Poor electrode contact of the current electrodes is of particular concern in relation to the use of the signal generator, for example. If contact impedance is very high as a result of poor contact, the signal generator may not be able to deliver the selected amount of current (if a current controlled mode is used), or the current delivered may be excessively low (if a voltage controlled mode is used).

Following from this, the configuration of the apparatus can be analysed based on the measured voltage (V(A-B)) or bioimpedance (Z(A-B)) between the first electrodes and/or based on the measured voltage (V(C-D)) or bioimpedance (Z(C-D)) between the second electrodes. Analysing the configuration may comprise determining if the voltage (V(A-B)) or bioimpedance (Z(A-B)) between the first sensing electrode (A) and the first current electrode (B) and/or the voltage (V(C-D)) or bioimpedance (Z(C-D)) between the second current electrode (C) and the second sensing electrode (D) is above or below a respective predetermined threshold.

A user may re-connect one or more of the electrodes to the tissue when the voltage/bioimpedance measurements (V(A-B), Z(A-B), V(C-D), Z(C-D)) are above a predetermined threshold, indicative of poor electrode contact. The apparatus may automatically issue an alarm or other alert signal to indicate when poor electrode contact has been determined based on these measurements.

As indicated above, bioimpedance may be determined by measuring the voltage (V(A-D) between the outer pair of sensing electrodes (A,D), e.g., using equation 1. However, in accordance with the first and second aspects, additionally or alternatively, voltage (V(A-C) or V(B-D)) may be measured between one of the outer pair of sensing electrodes (A,D) and the current electrode (B,C) that is on the opposite side of the region of interest from that sensing electrode, and voltage (V(C-D) or V(A-B)) may be measured between the other of the outer pair of sensing electrodes (A,D) and the current electrode (B,C) that is on the same side of the region of interest as that other sensing electrode, and bioimpedance (Z_{ROI}) across the tissue region of interest may be determined from the voltage measurements. In essence, this approach may allow bioimpedance to be measured across the tissue region of interest, between one of the sensing electrodes (A,D) and one of the current electrodes (B,C), while enabling the contribution towards this bioimpedance measurement of the transverse impedance beneath the current electrode (B,C) to be substantially factored out. In carrying out this “combinational two-electrode” approach, bioimpedance Z′_{ROI} Can be determined using equation 4a, and/or bioimpedance Z″_{ROI} can be determined using equation 4b.

$$\begin{matrix}{Z}_{ROI}^{′ } = \frac{V ( A - C )}{I ( t )} - \frac{V ( C - D )}{I ( t )} & [ 4 a ] \\ {Z}_{ROI}^{″} = \frac{V ( B - D )}{I ( t )} - \frac{V ( A - B )}{I ( t )} & [ 4 b ]\end{matrix}$$

In one embodiment, the monitoring device is also adapted to measure voltage V(B-C) between the inner pair of current electrodes (B,C), resulting from the application of the first electrical signal or a further electrical signal. In this embodiment, voltages (V(A-C), V(B-D)) are measured between each electrode of the outer pair of sensing electrodes (A,D) and the current electrode (B,C) that is on the opposite side of the region of interest from that sensing electrode, and bioimpedance (Z_{ROI}) across the tissue region of interest is determined from the voltage measurements. In carrying out this further “combinational two-electrode” approach, bioimpedance Z′″_{ROI} can be determined using equation 4c.

$${Z}_{ROI}^{′′′} = \frac{V ( A - C )}{I ( t )} + \frac{V ( B - D )}{I ( t )} - \frac{V ( B - C )}{I ( t )} \qquad [ 4 c ]$$

In general, the combinational two-electrode techniques may provide for wider assessment of the configuration of the apparatus. The technique may be performed automatically or semi-automatically by the apparatus and it may reduce or eliminate errors associated with standard tetrapolar electrode arrangements.

A comparison may be made between the two or three of the measured bioimpedances (Z′_{ROI}, Z″_{ROI}, Z′″_{ROI}) calculated in accordance with equations 4a, 4b and 4c.

The configuration of the apparatus may be assessed using equation 5:

$$\frac{V ( B - C )}{I ( t )} = \frac{V ( A - D )}{I ( t )} + \frac{V ( A - B )}{I ( t )} + \frac{V ( C - D )}{I ( t )} \qquad [ 5 ]$$

If equation 5 is not met, it may be determined that the sensing electrodes (A,D) are positioned either too close or too far from the current electrodes (B,C) to provide an accurate measurement of bioimpedance at the region of interest. More specifically, if the current electrodes (B,C) are positioned relatively close to each other and equation 5 is not met, the sensing electrodes (A,D) are likely to be too far away from the current electrodes (B,C). The apparatus may automatically issue an alarm or other alert signal to indicate that the sensing electrodes (A,D) are too far away from and/or to indicate that the sensing electrodes should be moved closer to the current electrodes (B,C). Alternatively, if the current electrodes (B,C) are positioned relatively remotely from each other and equation 5 is not met, the sensing electrodes (A,D) are likely to be too close to the current electrodes (B,C). The apparatus may automatically issue an alarm or other alert signal to indicate that the sensing electrodes (A,D) are too close to and/or to indicate that the sensing electrodes should be moved away from the current electrodes (B,C). This approach may apply where the electrodes are substantially the same size and where there is good electrode contact, e.g., as determined based on any one of equations 2 to 4b.

The apparatus may be pre-configured for use with only one of closely-spaced current electrodes and remotely-spaced current electrodes, and therefore may be preconfigured to always provide an indication, if equation 5 is not met, that the sensing electrodes are too close and/or should be moved away from the current electrodes, or always provide an indication that the sensing electrodes are too far away and/or should be moved towards the current electrodes. Alternatively, the apparatus may be adapted to receive an input signal indicative of whether the current electrodes are closely-spaced or remotely-spaced and/or receive a measurement of the spacing of these electrodes, and adapt accordingly its indication regarding whether or not the sensing electrodes are too close to or too far from and/or should be moved closer to or further away from the current electrodes. To the extent that equation 5 is not met, the quality of the apparatus configuration may be quantified based on the degree of divergence from equation 5.

Closely-spaced inner current electrodes (B,C) may be used when the ROI is superficial localized tissue. In this circumstance, as indicated above, the electrode arrangement may be such that the outer sensing electrodes (A,D) are positioned as close as possible to, but not touching, closely-spaced inner current electrodes (B,C). For example, the distance between the first electrodes (A,B) and/or the distance between the second electrodes (C,D) may be less than 50 mm, less than 20 mm, less than 10 mm, less than 5 mm, less than 2 mm or otherwise.

Remotely-spaced inner current electrodes (B,C) may be used when the ROI is non-localized tissue. In this circumstance, as indicated above, the electrode arrangement may be such that the outer sensing electrodes (A,D) are positioned relatively remotely from remotely-spaced inner current electrodes (B,C). For example, the distance between the first electrodes (A,B) and/or the distance between the second electrodes (C,D) may be greater than 50 mm, greater than 100 mm, greater than 200 mm or otherwise.

The first electrical signal may be a non-therapeutic (non-stimulating) electrical signal. Accordingly, the first electrical signal may be applied for the purposes of characterising body tissue only. However, alternatively, the first electrical signal may be a therapeutic (stimulating) electrical signal and/or a second electrical signal may be applied that is a therapeutic (stimulating) electrical signal. To provide a therapeutic, stimulating effect, the second electrical signal may have different characteristics to the first electrical signal. Thus, in one embodiment, a second electrical signal may be applied between the inner pair of current electrodes (B,C) at a different time from the application of the first electrical signal, wherein the second electrical signal typically has different characteristics to the first electrical signal. The therapeutic electrical signal may provide electrostimulation therapy to the tissue region of interest or otherwise.

According to a third aspect, the present disclosure provides a method for characterization of body tissue, the method comprising:

applying a first electrical signal between a pair of current electrodes (B′, D′), the pair of current electrodes comprising a first current electrode (B′) and a second current electrode (D′), the first current electrode (B′) being connected to the surface of a tissue region of interest and the second current electrode (D′) being connected to tissue at a second side of the tissue region of interest;

measuring one or more voltages between a first sensing electrode (A′) and a sensing electrode (C′) and/or a third sensing electrode (E′), and/or between one of the first, second and third sensing electrodes (A′, C′, E′) and one of the pair of current electrodes (B′, D′), resulting from the application of the first electrical signal, wherein the first sensing electrode (A′) is connected to tissue at a first side of the tissue region of interest, substantially opposite to the second side, and the second and/or third sensing electrodes (C′, E′) are connected to tissue at the second side of the tissue region of interest; and

determining, from the one or more voltage measurements, bioimpedance (Z_{ROI}) across the tissue region of interest.

In one embodiment, the second sensing electrode (C′) is connected to tissue between the first and second current electrodes (B′, D′) and the third sensing electrode (E′) is connected to tissue on an opposite side of the second current electrode (D′) to the tissue region of interest.

The method may employ similar techniques to characterise body tissue as described with respect to the first and second aspects, and can also allow characterisation of transverse impedances under the current electrodes. A primary difference, however, is that one of the current electrodes, the first current electrode (B′), is generally placed directly on top of the tissue region of interest, rather than to one side of the tissue region of interest. The first current electrode can extend across the surface of the entire tissue region of interest. A relatively large electrode such as this is known to be used in two-electrode stimulation systems, particularly where therapeutic electrostimulation of the tissue is carried out. However, the present disclosure provides for a particular configuration of additional electrodes that allow enhanced bioimpedance analysis of the region of interest (ROI) and characterisation of transverse impedances and hence quality of electrode contact.

The first electrical signal may be a non-therapeutic (non-stimulating) electrical signal. Accordingly, the first electrical signal may be applied for the purposes of characterising body tissue only. However, alternatively, the first electrical signal may be a therapeutic (stimulating) electrical signal and/or a second electrical signal may be applied that is a therapeutic (stimulating) electrical signal. To provide a therapeutic, stimulating effect, the second electrical signal may have different characteristics to the first electrical signal. Thus, in one embodiment, the method may comprise applying a second electrical signal between the pair of current electrodes (B′, D′) at a different time to the application of the first electrical signal, wherein the second electrical signal typically has different characteristics than the first electrical signal. The therapeutic electrical signal may provide electrostimulation therapy to the tissue region of interest or otherwise.

An apparatus that can be used to carry out the method described above is also disclosed herein. In particular, in accordance with a fourth aspect, the present disclosure provides apparatus for characterization of body tissue, the apparatus comprising:

a pair of current electrodes (B′, D′), the pair of current electrodes comprising a first current electrode (B′), adapted to connect to the surface of a tissue region of interest, and comprising a second current electrode (D′), adapted to connect to tissue at a second side of the tissue region of interest;

a signal generator adapted to apply a first electrical signal between the pair of current electrodes (B′, D′);

a plurality of sensing electrodes (A′, C′, E′), the sensing electrodes comprising a first sensing electrode (A′), adapted to connect to tissue at a first side of the tissue region of interest, substantially opposite to the second side, and comprising a second sensing electrode (C′) and/or a third sensing electrode (E′), adapted to connect to tissue at the second side of the tissue region of interest; and

a monitoring device adapted to measure one or more voltages between the first sensing electrode (A′) and the second and/or third sensing electrode (C′, E′) and/or between one of the sensing electrodes (A′, C′, E′) and one of the pair of current electrodes (B′, D′), resulting from the application of the first electrical signal, and determine, from the one or more voltage measurements, bioimpedance (Z_{ROI}) across the tissue region of interest.

The second sensing electrode (C′) is adapted to connect to tissue between the first and second current electrodes (B′, D′) and the third sensing electrode (E′), if provided, is adapted to connect to tissue on an opposite side of the second current electrode (D′) to the tissue region of interest.

In this portion of the summary section, letters in brackets, e.g. (A′, B′, C′, D′, E′), have been used to aid recognition of each electrode under discussion. To further aid recognition, reference may be made to FIG. 2, where corresponding lettering has been used, and which Figure is discussed in more detail further below. Nevertheless, it is not intended that the electrode arrangements disclosed in this summary section are limited to any arrangements shown in the Figures. The discussions in this summary section can be considered either in conjunction with, or entirely independently of, the Figures.

In accordance with the third and fourth aspects, in one embodiment, voltage (V(A′-C′)) is measured between the first and second sensing electrodes (A, C′) resulting from the application of the first electrical signal and bioimpedance (Z_{ROI}) across the tissue region of interest is determined from the voltage measurement (V(A′-C′).

Where voltage V(A′-C′) is measured between the first and second sensing electrodes (A′, C′), during application of the first electrical signal having a waveform or waveform spectrum suitable for electrical bioimpedance measurement (such as a controlled current AC waveform I(t)), the bioimpedance Z_{ROI} may be determined using equation 6:

$${Z}_{ROI} = \frac{V ( {A}^{′} - {C}^{′} )}{I ( t )} \qquad [ 6 ]$$

Through inclusion of phase-sensitive electronics in the apparatus, impedance Z can be resolved into real (resistive) and imaginary (reactive) components.

Characterisation of electrode-to-tissue contact at the first current electrode (B′) may be carried out by measuring a voltage (V(A′-B′)) between the first electrodes (A′, B′) resulting from the application of the first electrical signal or a further electrical signal applied between the pair of current electrodes (B′, D′), and optionally determining, from the voltage measurement (V(A′-B′)), bioimpedance (Z(A′-B′)) across tissue between the first electrodes (A′, B′).

Characterisation of electrode-to-tissue contact at the second current electrode (D′) may be carried out using the third sensing electrode. A voltage (V(D′-E′)) may be measured between the second current electrode (D′) and the third sensing electrode (E′), resulting from the application of the first electrical signal or a further electrical signal applied between the pair of current electrodes (B′, D′), and optionally bioimpedance (Z(D′-E′) may be determined from the voltage measurement (V(D′-E′)) across tissue between the second current electrode (D′) and the third sensing electrode (E′).

The bioimpedance measurements (Z(A′-B′), Z(D′-E′)) provide essentially the transverse impedances (Z_{B′}, Z_{D′}) beneath the first and second current electrodes (B′, D′), respectively. The transverse impedances (Z_{B′}, Z_{D′}) can be calculated using equations 7 and 8.

$$\begin{matrix}{Z}_{{B}^{′}} = \frac{V ( {A}^{′} - {B}^{′} )}{I ( t )} & [ 7 ] \\ {Z}_{{D}^{′}} = \frac{V ( {D}^{′} - {E}^{′} )}{I ( t )} & [ 8 ]\end{matrix}$$

The greater the voltage or bioimpedance measurements (V(A′-B′), Z(A′-B′), V(D′-E′), Z(D′-E′)), the greater the transverse impedances (Z_{B′}, Z_{B′}) at the current electrodes (B′, D′). Higher transverse impedances can indicate that there is poor electrode contact. Poor electrode contact of the current electrodes is of particular concern in relation to the use of the signal generator. If contact impedance is very high as a result of poor contact, the signal generator may not be able to deliver the selected amount of current (if a current controlled mode is used), or the current delivered may be excessively low (if a voltage controlled mode is used).

Following from this, the configuration of the bioimpedance apparatus may be analysed based on the measured voltage (V(A′-B′)) or bioimpedance (Z(A′-B′)) between the first sensing electrode (A′) and the first current electrode (B′) and/or based on the measured voltage (V(D′-E′)) or bioimpedance (Z(D′-E′)) between the second current electrode (D′) and the third sensing electrode (E′). Analysing the configuration may comprise determining if the voltage (V(A′-B′)) or bioimpedance (Z(A′-B′)) between the first sensing electrode (A′) and the first current electrode (B′) and/or the voltage (V(D′-E′)) or bioimpedance (Z(D′-E′)) between the second current electrode (D′) and the third sensing electrode (E′) is substantially above or below a respective predetermined threshold.

A user may re-connect one or more of the electrodes to the tissue when the voltage/bioimpedance measurements (V(A′-B′), Z(A′-B′), V(D′-E′), Z(D′-E′)) are above the predetermined threshold, indicative of poor electrode contact. The apparatus may automatically issue an alarm or other alert signal to indicate when poor electrode contact has been determined based on these measurements.

As indicated above, bioimpedance (Z_{ROI}) at the region of interest may be determined by measuring the voltage (V(A′-C′) between the pair of sensing electrodes (A′, C′), However, in accordance with the third and fourth aspects, additionally or alternatively, bioimpedance (Z′_{ROI}, Z″_{ROI}, Z′″_{ROI}) at the region of interest may be measured in a number of other ways.

For example, bioimpedance (Z_{ROI}) at the region of interest may be determined by measuring:

voltage (B′-C′) between the first current electrode (B′) and the second sensing electrode (C′) and voltage (V(A′-B′) between the first sensing electrode (A′) and the first current electrode (B′); and/or

voltage (A′-E′) between the first sensing electrode (A′) and the third sensing electrode (E′) and voltage (V(C′-E′) between the second sensing electrode (C′) and the third sensing electrode (E′); and/or

voltage (A′-D′) between the first sensing electrode (A′) and the second current electrode (D′) and voltage (V(C′-D′) between the second sensing electrode (C′) and the second current electrode (D′).

These measurements allow bioimpedance to be determined across a region that includes the tissue region of interest, while substantially allowing the contribution to this bioimpedance by tissue and/or electrodes, independent of the tissue region of interest, to be factored out. In carrying out this “combinational two-electrode” technique, bioimpedances Z′_{ROI}, Z″_{ROI}, Z′″_{ROI}) can be determined using equations 9a-9c. In general, the combinational two-electrode technique may provide for wider assessment of the configuration of the apparatus. The technique may be performed automatically or semi-automatically by the apparatus and it may reduce or eliminate errors associated with standard tetrapolar electrode arrangements.

$$\begin{matrix}{Z}_{ROI}^{′} = \frac{V ( {B}^{′} - {C}^{′} )}{I ( t )} - \frac{V ( {A}^{′} - {B}^{′} )}{I ( t )} & [ 9 a ] \\ {Z}_{ROI}^{″} = \frac{V ( {A}^{′} - {E}^{′} )}{I ( t )} - \frac{V ( {C}^{′} - {E}^{′} )}{I ( t )} & [ 9 b ] \\ {Z}_{ROI}^{′′′} = \frac{V ( {A}^{′} - {D}^{′} )}{I ( t )} - \frac{V ( {C}^{′} - {D}^{′} )}{I ( t )} & [ 9 c ]\end{matrix}$$

A comparison may be made between the two or more of the measured bioimpedances (Z′_{ROI}, Z″_{ROI}, Z′″_{RoI}) calculated in accordance with equations 9a to 9c.

The configuration of the apparatus may be assessed using equation 10:

$$\frac{V ( {B}^{′} - {D}^{′} )}{I ( t )} = \frac{V ( {A}^{′} - {C}^{′} )}{I ( t )} + \frac{V ( {A}^{′} - {B}^{′} )}{I ( t )} + \frac{V ( {D}^{′} - {E}^{′} )}{I ( t )} \qquad [ 10 ]$$

If equation 10 is not met, it may be determined that one or both of the first and second sensing electrodes (A′, C′) are positioned either too close or too far from the first current electrodes (B′) to provide an accurate measurement of bioimpedance at the region of interest. More specifically, if the current electrodes (B′, D′) are positioned relatively close to each other and equation 10 is not met, at least one of the first and second sensing electrodes (A′, C′) is likely to be too far away from the current electrodes (B′). The apparatus may automatically issue an alarm or other alert signal to indicate that the first and/or second sensing electrode (A′, C′) is too far away from and/or to indicate that the first and/or second sensing electrode (A′, C′) should be moved closer to the current electrodes (B′). Alternatively, if the current electrodes (B′, D′) are positioned relatively remotely from each other and equation 10 is not met, at least one of the first and second sensing electrodes (A′, C′) is likely to be too close to the first current electrode (B′). The apparatus may automatically issue an alarm or other alert signal to indicate that the first and/or second sensing electrode (A′, C′) is too close to and/or to indicate that the first and/or second sensing electrode should be moved away from the first current electrode (B′). This approach may apply where the electrodes are substantially the same size and where there is good electrode contact, e.g. as determined based on any one of equations 6 to 9c.

The electrode arrangement may be such that the second sensing electrode (C′) is as close as possible to, but not touching, the first current electrode (B′). For example, the distance between the electrodes (C′, B′) may be less than 50 mm, less than 20 mm, less than 10 mm, less than 5 mm, less than 2 mm or otherwise. Alternatively, the electrode arrangement may be such that the second sensing electrode (C′) is distant to the first current electrode (B′). For example, the distance between the electrodes (C′, B′) may be greater than 50 mm, greater than 100 mm, greater than 200 mm or otherwise. Further, the electrode arrangement may be such that the first sensing electrode (A′) is distant to the first current electrode (B′). For example, the distance between the electrodes (A′, B′) may be greater than 50 mm, greater than 100 mm, greater than 200 mm or otherwise.

The methods and apparatuses described herein may have a variety of applications. For example, they may be employed for general bioelectrical impedance analysis applications such as body composition determination, fluid management, wound assessment and monitoring, which may give more accurate impedance determinations than existing arrangements. For example, for subjects such as infants and children having small limbs it may be impossible to satisfy standard protocols for minimum spacing between current and voltage based on anatomical landmarks. Methods and apparatuses described herein, however, are not limited by any minimum spacings for accurate measurement. The methods and apparatuses may also be used in wound healing applications and muscle condition assessment and/or recovery applications or otherwise. In some embodiments, the region of interest may be a portion of the body that is defective. For example, the region of interest may be a wound, or a diseased or strained muscle or likewise.

The methods and apparatuses described herein may be particularly advantageous where localized, e.g. superficial, tissue is to be monitored. Since, at least with respect to the first and second aspects, the current electrodes can be placed as close as desired to the region of interest (e.g. a superficial wound), without needing to interpose a sensing electrode therebetween, the electrical signal for bioimpedance measurement can course through the localized region of interest, rather than deeper tissue.

In one embodiment, bioimpedance is measured, using the techniques described herein, on corresponding limbs. A relative bilateral bioimpedance data comparison may therefore be achieved. For example, bioimpedance measurements at a region of interest on one limb (e.g. the left arm or leg), which is associated with defected tissue such as a wound, may be compared with bioimpedance measurements at a region of interest on another limb (e.g. the right arm or leg), which is associated with normal, healthy tissue.

### BRIEF DESCRIPTION OF DRAWINGS

By way of example only, embodiments are now described with reference to the accompanying drawings, in which:

FIG. 1 provides a representation of electrode positioning in a method and apparatus for characterization of body tissue according to an embodiment of the present disclosure;

FIG. 2 provides a representation of electrode positioning in a method and apparatus for characterization of body tissue according to another embodiment of the present disclosure;

FIG. 3 shows a schematic illustration of apparatus for characterization of body tissue according to an embodiment of the present disclosure;

FIG. 4 shows a schematic illustration of apparatus for characterization of body tissue according to another embodiment of the present disclosure;

FIG. 5 shows a representation of a body upon which bioimpedance measurements are carried out on opposing limbs for comparative purposes;

FIGS. 6a and 6b provide schematic illustrations of a standard four-electrode (tetrapolar) arrangement and a reciprocal four-electrode arrangement, respectively, as used in computer modelling studies according to the present disclosure; and

FIG. 7 provides a representation of a tissue/electrode model with selected dimensions for the computer modelling studies.

### DESCRIPTION OF EMBODIMENTS

In a method and an apparatus for characterization of body tissue according to an embodiment of the present disclosure, electrodes are provided in accordance with the arrangement shown in FIG. 1. In particular, at least four electrodes are provided (described for ease of reference as electrodes A, B, C, D) which are in electrical contact with a surface 11 of tissue 10 of a patient. The inner two current electrodes B, C are adapted to apply an electrical signal from a signal generator 20 to the tissue 10. The outer two sensing electrodes A, D are provided for the purpose of sensing voltages. Nonetheless, the current electrodes B, C can also provide a voltage sensing function.

Application of the electrical signal to the tissue permits measurement of bioimpedance Z_{ROI} across a region of interest (ROI) 12 of the tissue 10. In this embodiment, the region of interest 12 is positioned at and directly underneath the tissue surface 11, between the inner pair of current electrodes B, C.

To measure the bioimpedance Z_{ROI} across the region of interest, the voltage V(A-D), is measured between the outer pair of sensing electrodes A, D during application of the electrical signal. Since current is applied by the current electrodes B, C through substantially the region of interest 12 only, the voltage measurement is dictated by the impedance of the tissue 10 at the region of interest 12, and is substantially independent of the impedance of the tissue 10 outside of the region of interest 12. Accordingly, through application of the electrical signal having a waveform or waveform spectrum suitable for electrical bioimpedance measurement (such as a controlled current AC waveform I(t)), the bioimpedance Z_{ROI} may be determined using equation 1:

$${Z}_{ROI} = \frac{V ( A - D )}{I ( t )} \qquad [ 1 ]$$

The arrangement can provide an accurate indication of bioimpedance at the region of interest 12 located between the inner current electrodes B, C. Moreover, the arrangement allows for characterisation of the quality of electrode-to-tissue contact at the current electrodes B, C.

In more detail, transverse impedances Z_{B}, Z_{C} at the contact positions between the current electrodes B, C and the tissue surface can be calculated by measuring the voltage V(A-B) between the first sensing electrode A and the first current electrode B, and by measuring the voltage V(C-D) between the second current electrode C and the second sensing electrode D. The transverse impedances (Z_{B}, Z_{C}) can be calculated using equations 2 and 3.

$$\begin{matrix}{Z}_{B} = \frac{V ( A - B )}{I ( t )} & [ 2 ] \\ {Z}_{C} = \frac{V ( C - D )}{I ( t )} & [ 3 ]\end{matrix}$$

Equations 2 and 3 are used to obtain the transverse impedances since current is applied between current electrodes B and C only, and therefore the voltage measurements V(A-B), V(C-D) are dictated by the impedance at the contact position between the current electrodes B, C and the tissue surface (e.g. skin), substantially independently of the impedance of remaining tissue between the sensing electrodes A, D and the respective current electrodes B, C.

If there is poor electrode contact, the transverse impedances Z_{B} and Z_{C} will be above respective predetermined thresholds.

In this or alternative embodiments, bioimpedance is also estimated by measuring voltage V(A-C) or V(B-D) between one of the outer pair of sensing electrodes A,D and the current electrode B,C that is on the opposite side of the region of interest from that sensing electrode, and measuring voltage V(C-D) or V(A-B) between the other of the outer pair of sensing electrodes A,D and the current electrode that is on the same side of the region of interest as that other sensing electrode.

Using these “combinational two-electrode” techniques, bioimpedance Z′_{ROI} and/or Z″_{ROI} is determined using equation 4a and/or equation 4b. In general, the combinational two-electrode techniques may provide for wider assessment of the configuration of the apparatus.

$$\begin{matrix}{Z}_{ROI}^{′} = \frac{V ( A - C )}{I ( t )} - \frac{V ( C - D )}{I ( t )} & [ 4 a ] \\ {Z}_{ROI}^{″} = \frac{V ( B - D )}{I ( t )} - \frac{V ( A - B )}{I ( t )} & [ 4 b ]\end{matrix}$$

In an alternative embodiment, voltage V(B-C) is measured between the inner pair of current electrodes (B,C). In this alternative embodiment, voltages (V(A-C), V(B-D)) are also measured between each electrode of the outer pair of sensing electrodes (A,D) and the current electrode (B,C) that is on the opposite side of the region of interest from that sensing electrode, and bioimpedance (Z′″_{ROI}) across the tissue region of interest is determined from the voltage measurements. In carrying out this further combinational two-electrode approach, bioimpedance Z′″_{ROI} can be determined using equation 4c.

$${Z}_{ROI}^{′′′} = \frac{V ( A - C )}{I ( t )} + \frac{V ( B - D )}{I ( t )} - \frac{V ( B - C )}{I ( t )} \qquad [ 4 c ]$$

The voltage measurements are also used in combination to provide a wider assessment of the configuration of the apparatus.

The configuration of the apparatus can be assessed using equation 5:

$$\frac{V ( B - C )}{I ( t )} = \frac{V ( A - D )}{I ( t )} + \frac{V ( A - B )}{I ( t )} + \frac{V ( C - D )}{I ( t )} \qquad [ 5 ]$$

If equation 5 is not met, it can be determined in this embodiment, where the region of interest is relatively localized tissue and the current electrodes are placed relatively closely, that the sensing electrodes A, D are positioned too far from the current electrodes (B,C) to provide an accurate measurement of bioimpedance at the region of interest. In alternative embodiments, if the current electrodes B, C are positioned relatively remotely, to monitor non-localized tissue, and equation 5 is not met, the sensing electrodes A, D are likely to be too close to the current electrodes B, C. This assessment applies where the electrodes are substantially the same size and where there is good electrode contact, e.g. as determined based on any one of equations 2 to 4c.

As indicated, in this embodiment, the ROI 12 is superficial localized tissue. The electrode arrangement is therefore such that the outer sensing electrodes A, D are as close as possible to, but not touching, the closest inner current electrodes B, C (the electrode positioning in FIG. 1 is not drawn to scale). In particular, the outer sensing electrodes A, D are separated by less than 50 mm from the closest inner current electrodes B, C, e.g. less than 20 mm, less than 10 mm, less than 5 mm or less than 2 mm.

In alternative embodiments, the electrode arrangement can be such that the outer sensing electrodes A, D are more remote (e.g. greater than 50 mm) from the inner current electrodes B, C, particular where the ROI is non-localised tissue.

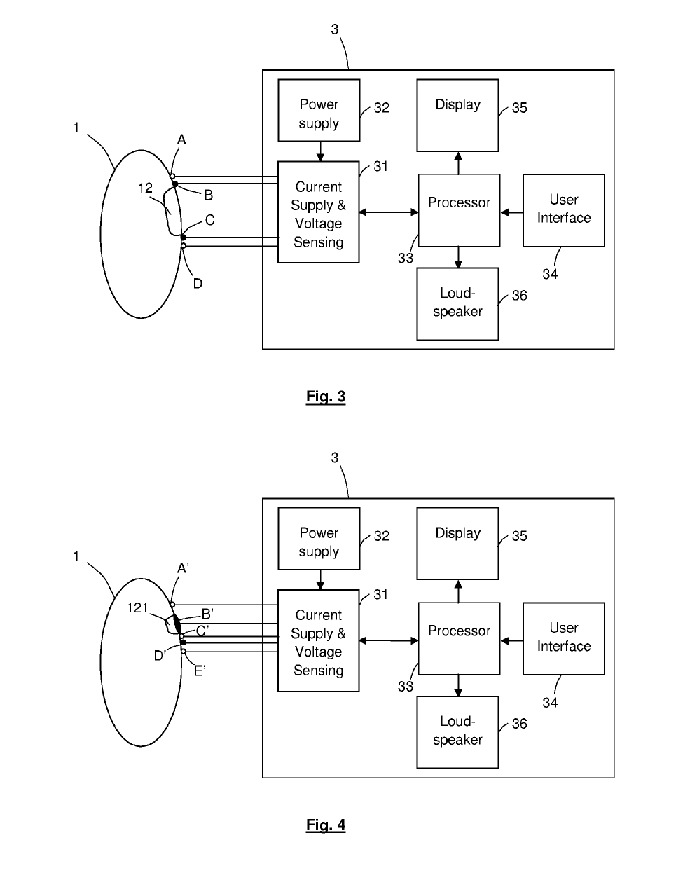

Apparatus employing an electrode configuration in accordance with FIG. 1, and utilising the above-described methods for determining impedances, is shown schematically in FIG. 3.

The apparatus comprises integrated drive and voltage sensing circuitry 31, the circuitry 31 being connected to the inner pair of current electrodes B, C to deliver electrical signal current from a power supply 32 across a tissue region of interest 12 of a patient 1 and to sense voltages between electrodes while the signal is delivered. The drive and voltage circuitry 31 is connected to a processor 33, which provides a monitoring device and is configured to control the delivery of the electrical signal, determine the voltage measurements, and process the measurements to determine one or more bioimpedance measurement Z_{RoI}, Z′_{ROI}, Z″_{ROI}, Z′″_{ROI} across the region of interest in accordance with equations 1, 4a, 4b and 4c, and/or one or more transverse bioimpedances Z_{C}, Z_{D} in accordance with equations 2 and 3, and/or to assess the configuration of the apparatus in accordance with equation 5.

A user interface 34 (e.g. keyboard, touch screen, etc.) is connected to the processer 33 to allow the user to start and stop the process and/or control other characteristics of the process. The processor 33 is also connected to a display 35 to display at least the determined bioimpedance(s) and/or transverse bioimpedances for the region of interest and/or other indicators about the configuration of the apparatus. Particularly where a touch screen is employed, the display and user interface may be provided by substantially the same element. The processor 33 is also connected to a loudspeaker 36 to provide an alert signal when either one of the transverse impedances Z_{B}, Z_{C} (and/or the voltage measurements V(A-B), V(C-D) that can be used to determine the transverse impedances Z_{B}, Z_{C}) are below respective predetermined thresholds. The predetermined thresholds can be adapted for different electrode sizes. The alert signal from the loudspeaker 36 provides an indication to the user of poor electrode contact. An alert may additionally, or alternatively, be issued on the display 35.

In this embodiment, the circuitry 31, power supply 32, processor 33, user interface 34, display, 35, and loud-speaker 36 are integrated into a single bioimpedance analysis unit 3, manufactured to carry out the process described above However, any one or more of these components may be located separately and connected by wires and/or other appropriate communication links. For example, the processor 33, user interface 34, loud-speaker 36 and display 35 may be provided by more standard personal computing apparatus configured to run bespoke software in order to implement the process described above, which computing apparatus is connected to the integrated drive and voltage sensing circuitry 31 and power supply. In general, a wide variety of apparatus configurations may be used in order to carry out the process described above.

Poor electrode contact of the current electrodes is of particular concern in relation to the use of the signal generator. If contact impedance is very high as a result of poor contact, the signal generator may not be able to deliver the selected amount of current (if a current controlled mode is used), or the current delivered may be excessively low (if a voltage controlled mode is used). Where poor electrode contact is determined, the processor 33 controls the circuitry 31 to cut off and/or adjust the electrical signal.

In this embodiment, the electrical signal is applied for non-therapeutic effect. That is, the electrical signal is applied for the purpose of analysing bioimpedance. However, the apparatus may be adapted such that the same or additional electrical signals are applied by the current electrodes for therapeutic purposes. These signals may have very different characteristics to those required for bioimpedance analysis, in order to provide therapeutically relevant electrostimulation of the tissue.

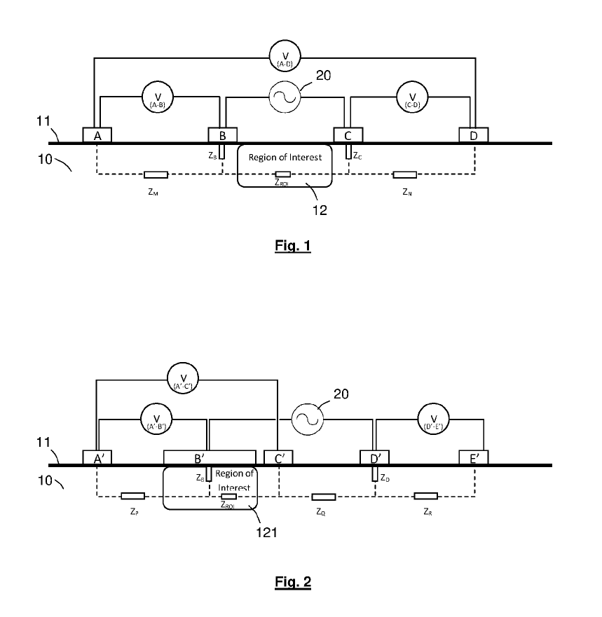

In a method and an apparatus for characterization of body tissue according to another embodiment of the present disclosure, electrodes are provided in accordance with the arrangement shown in FIG. 2. In particular, at least five electrodes are provided (described for ease of reference as electrodes A′, B′, C′, D′, E′), which are in electrical contact with a surface 11 of tissue 10 of a patient. A first current electrode B′ is connected to the surface 11 above a tissue region of interest 121. A second current electrode D′ is connected to the surface 11 on a second side of the tissue region of interest 121. A first sensing electrode A′ is connected to the surface 11 at an opposite, first side of the tissue region of interest from the second current electrode D′. A second sensing electrode C′ is connected to the surface 11 on the second side of the tissue region of interest and between the first and second current electrodes B′, D′. A third sensing electrode E′ is connected to the tissue 11 on the second side of the tissue region of interest but substantially an opposite side of the second current electrode (D′) to the tissue region of interest. The first and second current electrodes B′, D′ are adapted to apply an electrical signal from a signal generator 20 to the tissue 10. The sensing electrodes A′, C′, E′ are provided for the purpose of sensing voltages. Nonetheless, the current electrodes B′, D′ can also provide a voltage sensing function.

The first current electrode B′ differs from the second current electrode D′ by being configured to contact and extend over the surface of the tissue region of interest 121. The tissue region of interest 121 is therefore positioned directly underneath the first current electrode B′. A relatively large electrode such as this is known to be used in two-electrode stimulation systems, particularly where therapeutic electrostimulation of the tissue is carried out. However, the present disclosure provides additional electrodes A′, C′ and E′ for enhanced bioimpedance analysis and characterisation of electrode-to-tissue contact.

Application of the electrical signal to the tissue permits measurement of bioimpedance Z_{ROI} across the region of interest (ROI) 121 of the tissue. To measure the bioimpedance Z_{ROI} across the region of interest, voltage V(A′-C′) can be measured between the first and second sensing electrodes A′, C′ during application of the electrical signal. The second current electrode C′ is placed very close to, but not touching, the region of interest. Since the voltage measured between the first and second sensing electrodes A′, C′ is therefore dictated by the portion of the electrical current between the current electrodes B′, D′ that is substantially in the region of interest only, bioimpedance at the region of interest can be determined from the voltage V(A′-C′). Through application of the electrical signal having a waveform or waveform spectrum suitable for electrical bioimpedance measurement (such as a controlled current AC waveform I(t)), the bioimpedance Z_{ROI} can be determined using equation 6:

$${Z}_{ROI} = \frac{V ( {A}^{′} - {C}^{′} )}{I ( t )} \qquad [ 6 ]$$

The arrangement can provide an accurate indication of bioimpedance at the region of interest 121 between the current electrodes B′, D. Moreover, the arrangement allows for characterisation of electrode-to-tissue contact at the current electrodes B′, D′.

In more detail, transverse impedances Z_{B′}, Z_{D′} at the contact positions between the current electrodes B′, D′ and the tissue surface 11 can be calculated by measuring the voltage V(A′-B′) between the first sensing electrode A′ and the first current electrode B′, and by measuring the voltage V(D′-E′) between the second current electrode D′ and the third sensing electrode E′. The transverse impedances (Z_{B′}, Z_{D′}) can be calculated using equations 7 and 8.

$$\begin{matrix}{Z}_{{B}^{′}} = \frac{V ( {A}^{′} - {B}^{′} )}{I ( t )} & [ 7 ] \\ {Z}_{{D}^{′}} = \frac{V ( {D}^{′} - {E}^{′} )}{I ( t )} & [ 8 ]\end{matrix}$$

Equations 7 and 8 can be used to obtain the transverse impedances since current is applied between electrodes B′ and D′ only, and therefore the voltage measurements V(A′-B′), V(D′-E′) are dictated by the impedance beneath current electrodes B′, D′, substantially independently of the impedance of the remaining tissue between the current electrodes B′, D′ and the respective sensing electrodes A′, D′.

If there is poor electrode contact, the transverse impedances Z_{B′} and Z_{D′} will be above respective predetermined thresholds.

In this or alternative embodiments, bioimpedance at the region of interest may be measured in a number of additional or alternative ways

For example, bioimpedance Z′_{ROI}, Z″_{ROI}, Z′″_{ROI} at the region of interest may be determined by measuring:

voltage V(B′-C′) between the first current electrode B′ and the second sensing electrode C′ and voltage V(A′-B′) between the first sensing electrode A′ and the first current electrode B′; and/or

voltage V(A′-E′) between the first sensing electrode A′ and the third sensing electrode E′ and voltage V(C′-E′) between the second sensing electrode C′ and the third sensing electrode (E′); and/or

voltage V(A′-D′) between the first sensing electrode A′ and the second current electrode D′ and voltage V(C′-D′) between the second sensing electrode C′ and the second current electrode D′.

Using these “combinational two-electrode” techniques, bioimpedances Z′_{ROI}, Z″_{ROI}, Z′″_{ROI} can be determined using equations 9a-9c. In general, the combinational two-electrode techniques may provide for wider assessment of the configuration of the apparatus.

$$\begin{matrix}{Z}_{ROI}^{′} = \frac{V ( {B}^{′} - {C}^{′} )}{I ( t )} - \frac{V ( {A}^{′} - {B}^{′} )}{I ( t )} & [ 9 a ] \\ {Z}_{ROI}^{″} = \frac{V ( {A}^{′} - {E}^{′} )}{I ( t )} - \frac{V ( {C}^{′} - {E}^{′} )}{I ( t )} & [ 9 b ] \\ {Z}_{ROI}^{′′′} = \frac{V ( {A}^{′} - {D}^{′} )}{I ( t )} - \frac{V ( {C}^{′} - {D}^{′} )}{I ( t )} & [ 9 c ]\end{matrix}$$

The configuration of the apparatus can be assessed using equation 10:

$$\frac{V ( {B}^{′} - {D}^{′} )}{I ( t )} = \frac{V ( {A}^{′} - {C}^{′} )}{I ( t )} + \frac{V ( {A}^{′} - {B}^{′} )}{I ( t )} + \frac{V ( {D}^{′} - {E}^{′} )}{I ( t )} \qquad [ 10 ]$$

If equation 10 is not met, it can be determined in this embodiment, where the region of interest is relatively localized tissue and the current electrodes B′, D′ are placed relatively closely, that at least one of the first and second sensing electrodes A′, C′ is positioned too far from the first current electrode B′ to provide an accurate measurement of bioimpedance at the region of interest. In alternative embodiments, if the current electrodes B′, D′ are positioned relatively remotely, to monitor non-localized tissue, and equation 10 is not met, at least one of the first and second sensing electrodes A′, C′ is likely to be too close to the current electrodes B′, D′. This assessment applies where the electrodes are substantially the same size and where there is good electrode contact, e.g. as determined based on any one of equations 6 to 9c.

In this embodiment, with second current electrode D′ positioned remote to first current electrode B′, the second sensing electrode C′ is positioned as close as possible to, but not touching, the first current electrode B′. The distance between the electrodes B′ and C′ is less than 50 mm. On the other hand, the first sensing electrode A′ is positioned distant to the first current electrode B′. The distance between the electrodes A′ and B′ is greater than 50 mm. Also, the third sensing electrode E′ is positioned distant to the second current electrode D′. The distance between the electrodes D′ and E′ is greater than 50 mm.

Apparatus employing an electrode configuration in accordance with FIG. 2, and utilising the above-described methods for determining impedances, is shown schematically in FIG. 4. The apparatus is very similar to that described with respect to FIG. 3, and the same or very similar components have therefore been given the same reference numerals.

In this embodiment, however, the processor 33 is configured to process measurements to determine bioimpedance in accordance with any one or more of equations 6 to 9c, above. The processor causes the display 35 to display one or more bioimpedances Z_{ROI}, Z′_{ROI}, Z″_{ROI}, Z′″_{ROI} for the region of interest, determined using one or more of equations 6 and 9a to 9c, and/or display the transverse impedances Z_{B′} Z_{D′} determined using equations 7 and 8 and/or display indicators obtained during an assessment of the configuration of the apparatus determined using equation 10.

In contrast to the preceding embodiment, an electrical signal is applied by the current electrodes B′ D′ to achieve a therapeutic effect. That is, an electrical signal is applied not merely for the purpose of analysing bioimpedance. Bioimpedance may be analysed from the electrical signal providing the therapeutic effect, or from an additional electrical signal that has different and non-stimulating characteristics.

The embodiments described herein can be employed for general bioelectrical impedance analysis determination, which may give more accurate impedance determination especially for subjects such as infants and children having small limbs. In such cases, it may be impossible to satisfy standard protocols for minimum spacing between current and voltage based on anatomical landmarks. Methods and apparatuses described herein, however, do not require such minimum spacings for accurate measurement. The analysis can be performed before, during, after or independently of any therapeutic electrostimulation. The embodiments can also be used in wound healing applications and muscle condition assessment and recovery applications.

The embodiments described herein may be particularly advantageous where localized, e.g. superficial, tissue is to be monitored. Since, for example, in the embodiment of FIG. 1, the current electrodes can be placed as close as desired to the region of interest (e.g. a superficial wound), without needing to interpose a sensing electrode therebetween, the electrical signal for bioimpedance measurement can course through the localized region of interest, rather than deeper tissue.

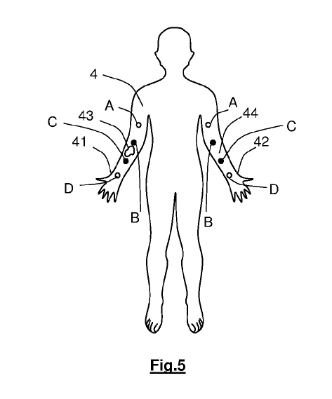

Referring to FIG. 5, in one embodiment, the techniques describe above with reference to FIGS. 1 and 3, are carried out on both a right arm 41 and a left arm 42 of a body 4. In this embodiment, the right arm has a tissue wound 43, and the wound is the region of interest. On the other hand, the left arm has no wound, and includes only healthy tissue at a region of interest 44. The configuration of the electrodes A, B, C, D on the left arm is essentially a mirror image of the configuration on the right arm. Accordingly, bioimpedance measurements at a region of interest, in particular the wound 43, of the right arm can be compared with bioimpedance measurements at the corresponding healthy region of interest 44 of the left arm 42. In alternative embodiments, the techniques described above with reference to FIGS. 2 and 4 can be applied to a similar bilateral bioimpedance measurement technique.

In any embodiment described herein, one or more of the current and sensing electrodes may use a wet-type contact (e.g. using a conductive paste or hydrogel etc.). The contact may be adhesive or non-adhesive. Alternatively or additionally, any one or more of the current and sensing electrodes may use a dry-type contact (e.g. using metal, metal oxide, conductive textile, conformal “tattoo-like” thin-film, microstructured carbon or ultrafine microneedle arrays etc.). Any one of more of the electrodes can be active electrodes which have small or unit amplification close to the electrode. This may allow the electrodes to be used without electrode gel, for example. Any one or more of the electrodes may rely on an adhesive contact with the patient, and/or tattoo-like van der Waal's contact and/or may be held in position using straps, bands, gloves, socks or belts or patient pressure (e.g. through a patient gripping or standing or resting on the electrodes).

Any one more of the electrodes may be fixed to the patient or be moveable. Any one or more of the electrodes may be provided by the same or different moveable probes, which are brought into contact with the patient.

Any one or more of the electrodes may take the form of metal plates, discs, strips, ellipses, heart-shapes, squares, rectangles or otherwise. Arrays of such electrodes may also be employed. The electrodes may have a width or diameter of between 0.1 and 15 mm, between 2 mm and 10 mm, between 10 mm and 20 mm, between 10 mm and 100 mm or otherwise.

Electrodes can be independently mounted, or two or more electrodes can be fabricated onto/into a single carrier material such as a dressing. All or part of any one of the electrodes may be disposable, and discarded following testing to reduce the likelihood of cross-contamination between patients. Alternatively, any one or more of the electrodes may be disinfected after use, and suitably dried.

Physical contact is preferably avoided between the examiner and the patient during measurement to prevent the introduction of short-circuit contributions into the electrical measurement. The examiner may wear insulating gloves to prevent this possibility.

Standard medically-approved leads and cables may be used to connect the electrodes to the control apparatus. The leads may be directly connected to the control apparatus or connected to a wireless transmission unit for wireless transfer of data and/or electrical signals.

The electrical signals for the non-therapeutic electrical characterisation of the body tissue may have a variety of different waveforms (frequencies, current levels) etc. The electrical signals may be a continuous AC waveform or pulsed. The electrical signals may have a frequency range of 1 kHz to 100 MHz, preferably 3 kHz to 1 MHz. Signals may be in the form of a single frequency, a set of frequencies (i.e. multi-frequency) or a continuous sweep (spectrum) of frequencies. For controlled current drive, applied current may be between 0.2 μA and 2 mA, e.g., between 5 μA and 250 μA or between 5 μA and 500 μA, or otherwise. For controlled voltage drive, the applied voltage may be between 0.05V to 5.0 V, e.g. between 0.2 V to 2.0 V, or otherwise. A constant current drive may be preferable to counteract slight variations in the surface profile/quality of electrode contact at the connection positions. The electrical signals for the therapeutic (stimulating) treatment of the body tissue can be direct current (DC) and/or alternating current (AC). The therapeutic schemes include constant DCs, DC pulses, and ACs. A significant number of choices of different amplitudes, frequencies (AC and pulsed DC), duty cycles, durations, current strengths, etc. can be used. Current is typically very small for DC stimulation (hundreds of A). Low-voltage pulsed currents can be pulses with durations up to 1 s and voltages up to 150 V, for example. Monophasic and biphasic pulsed currents can be low voltage and high voltage up to several hundred volts with short duration (cc s), for example.

### Computer Modelling Studies

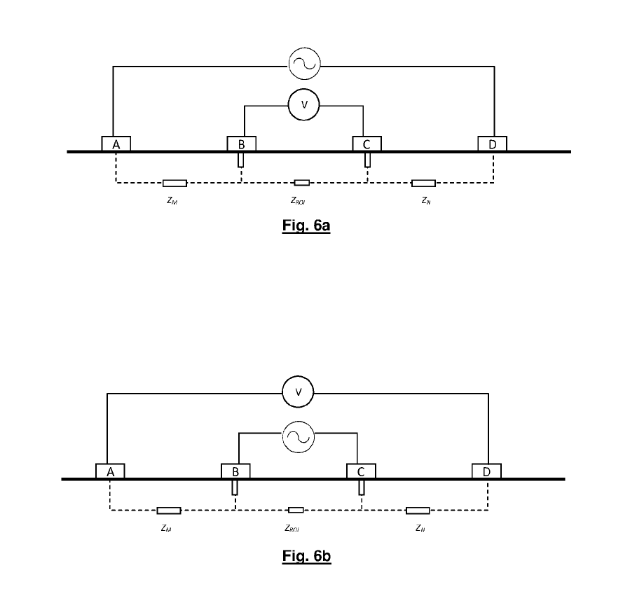

Finite Element Modelling (FEM) studies were conducted. The studies included comparisons of a four-electrode (tetrapolar) arrangement according to the present disclosure (“reciprocal arrangement”) with a standard four-electrode arrangement (“standard arrangement”). The reciprocal arrangement utilised an inner pair of current electrodes B, C and an outer pair of voltage sensing electrodes A, D, as represented schematically in FIG. 6a. The standard arrangement utilised an outer pair of current electrodes A, D and an inner pair of voltage sensing electrodes B, C as represented schematically in FIG. 6b.

The FEM studies were carried out using COMSOL Multiphysics v4.3a software.

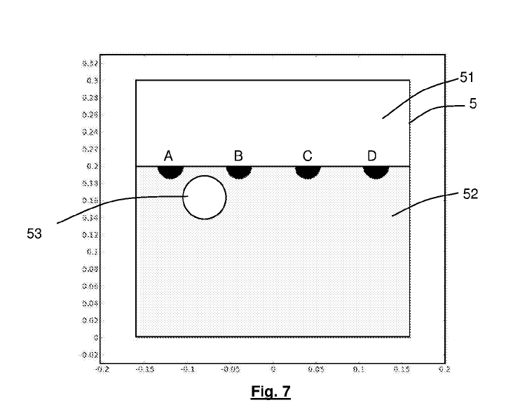

With reference to FIG. 7, a tissue/electrode model 5 was selected having dimensions replicating the geometry ratios used in Grimnes, S. and Matinsen, O. G. “Sources of error in tetrapolar impedance measurements on biomaterials and other ionic conductors”: J. Phys, D: Appl. Phys. 40 (2007), 9-14. The model included an air space 51 and tissue 52. A cylindrical object 53 was positioned between one of the outermost pairs of electrodes A and the adjacent inner electrode B to facilitate alteration of the impedance in the region between those electrodes. Specific dimensions were as follows: model domain width: 32.0 cm; air space height: 10.0 cm; tissue height: 20.0 cm; electrode radius: 1.5 cm; spacing between adjacent electrodes: 8.0 cm; cylindrical object radius: 2.5 cm.

The tissue 52 was chosen to have electrical properties of muscle and the cylindrical object 53 was chosen to have electrical properties of fat, in order to approximate an anticipated environment for use of the electrode arrangements. Fat was selected to characterise the cylindrical object as it has markedly lower conductivity and permittivity than muscle tissue (by one and two orders of magnitude, respectively) and thus would be expected to have a measurable effect on impedance in the vicinity of the object. The material properties were specifically as set forth in Table 1 below.

TABLE 1

| | | Relative |
|---|---|---|
| Material | Conductivity | permittivity |
| Tissue (muscle) | 0.36 [S/m] | 8,100 |
| Object (fat) | 0.03 [S/m] | 95 |
| Electrodes | 1E+07 [S/m] | 1 |
| Air | 1E+06 [S/m] | 1 |

The computational mesh was defined as being ‘physics-controlled’ with ‘normal’ element size. Two iterations of automatic mesh adaptation/refinement were carried out. The final mesh contained approximately 16,000 elements.

Current was applied to a flat external surface of one of the pair of current electrodes using the ‘Terminal’ boundary type, with the other of the current electrodes defined using the ‘Ground’ boundary type (y=0). Voltage sensing electrodes were defined using the ‘floating potential’ boundary type with zero current. The current was set at 100 μA with 100 kHz frequency.

### Study 1

Study 1 sought to compare the accuracy of estimation of Z_{ROI}, as determined using the reciprocal electrode arrangement using the measurement V(A-D)/I, with an estimation of Z_{ROI}, as determined using the standard electrode arrangement using the measurement V(B-C)/I. Study 1 also sought to determine if there were any increased negativity sensitivity error associated with impedance Z_{M} between electrodes A and B, and impedance Z_{N} between electrodes C and D, respectively.

For both the standard and reciprocal electrode arrangement, a cylindrical object was defined between electrodes A and B. The object was centred horizontally between the two electrodes and 1.5 times the object radius below the surface. Two scenarios were then considered for each electrode arrangement. In the first, the object was defined to have the same material properties as the surrounding tissue. In the second scenario the object was defined to have conductivity and permittivity smaller than the surrounding tissue (by one and two orders of magnitude, respectively). This two-domain approach was adopted for both electrode arrangements to ensure that the FEA mesh would be consistent between them, eliminating any effects which may have been attributed to different FEA meshes. The absence or presence of the object, and associated variation in Zₘ, was then used to assess the influence of negative sensitivity on the estimation of Z_{ROI}.

The results of the study are presented in Table 2 (standard electrode arrangement) and Table 3 (reciprocal electrode arrangement). With reference to Table 2 it can be seen that Z_{ROI} as measured using the standard electrode arrangement is estimated to be 2.5% lower when the object is present compared to that estimated for the homogeneous tissue scenario. This reduction is comprised of a 2.5% reduction in the real component and 2.0% reduction in the imaginary component of the impedance. Similarly, with reference to Table 3, it can be seen that Z_{ROI} as measured using the reciprocal electrode arrangement, is estimated to be 2.5% lower when the object is present compared to that estimated for the homogeneous tissue scenario. This reduction is again comprised of a 2.5% reduction in the real component and 2.0% reduction in the imaginary component of the impedance.

TABLE 2

| | All ′muscle′ | | Including ′fat′ object | |
|---|---|---|---|---|
| | R | Xi | R | Xi |
| Electrode A | 5.9763E−04 | −7.4820E−05 | 6.8679E−04 | −8.4108E−05 |
| (current source) | | | | |
| (V) | | | | |
| Electrode B | 3.7427E−04 | −4.6860E−05 | 3.6853E−04 | −4.6288E−05 |
| (voltage PU1) | | | | |
| (V) | | | | |
| Electrode C | 2.2340E−04 | −2.7971E−05 | 2.2138E−04 | −2.7766E−05 |
| (voltage PU2) | | | | |
| (V) | | | | |
| Electrode D | 0 | 0 | 0 | 0 |
| (ground) (V) | | | | |
| Z_{ROI} = | 1.5087 | −0.1889 | 1.4714 | −0.1852 |
| V(B − C)/I (Ω) | | | | |

TABLE 3

| | All ′muscle′ | | Including ′fat′ object | |
|---|---|---|---|---|
| | R | Xi | R | Xi |
| Electrode A | 2.2338E−04 | −2.7965E−05 | 2.1772E−04 | −2.7394E−05 |
| (voltage PU1) | | | | |
| (V) | | | | |
| Electrode B | 2.9587E−04 | −3.7039E−05 | 3.0733E−04 | −3.8256E−05 |
| (current source) | | | | |
| (V) | | | | |
| Electrode C | 0 | 0 | 0 | 0 |
| (ground) (V) | | | | |
| Electrode D | 7.2516E−05 | −9.0792E−06 | 7.0587E−05 | −8.8749E−06 |
| (voltage PU2) | | | | |
| (V) | | | | |
| Z_{ROI} = | 1.5086 | −0.1889 | 1.4713 | −0.1852 |
| V(A − D)/I (Ω) | | | | |

Thus, in Study 1 Z_{ROI} is estimated to be the same using both the standard and reciprocal electrode arrangements. Measurement of Z_{ROI} using the reciprocal electrode arrangement is equally as accurate as measurement of Z_{ROI} using the standard electrode arrangement. Further, there is no increased negativity sensitivity error associated with impedance Zₘ between electrodes A and B (and impedance Zₙ between electrodes C and D), when using the reciprocal electrode arrangement over the standard electrode arrangement.

### Study 2

Study 2 sought to compare the accuracy of estimation of Z′_{ROI}, Z″_{ROI} and Z′″_{ROI}, as determined using the reciprocal electrode arrangement using “combinational two electrode” measurement techniques, in accordance with equations 4a, 4b and 4c below, with the estimation of Z_{ROI} using the reciprocal electrode arrangement obtained in Study 1.

$$\begin{matrix}{Z}_{ROI}^{′} = \frac{V ( A - C )}{I ( t )} - \frac{V ( C - D )}{I ( t )} & [ 4 a ] \\ {Z}_{ROI}^{″} = \frac{V ( B - D )}{I ( t )} - \frac{V ( A - B )}{I ( t )} & [ 4 b ] \\ {Z}_{ROI}^{′′′} = \frac{V ( A - C )}{I ( t )} + \frac{V ( B - D )}{I ( t )} - \frac{V ( B - C )}{I ( t )} & [ 4 c ]\end{matrix}$$

The results of the study are presented in Table 4, in which it can be seen that the model predicts bioimpedance values for each combinational two electrode measurement technique that are identical to the bioimpedance values obtained in Study 1.

TABLE 4

| | All ′muscle′ | | Including ′fat′ object | |
|---|---|---|---|---|
| | R | Xi | R | Xi |
| Z_{ROI} = V(A − D)/I | 1.5086 | −0.1889 | 1.4713 | −0.1852 |
| Z’_{ROI} = V(A − C)/I − V(C − D)/I | 1.5086 | −0.1889 | 1.4713 | −0.1852 |
| Z”_{ROI} = V(B − D)/I − V(A − B)/1 | 1.5086 | −0.1889 | 1.4713 | −0.1852 |
| Z’’’_{ROI} = V(A − C)/I + V( B − D)/ | 1.5086 | −0.1889 | 1.4713 | −0.1852 |
| I − V(B − C)/I | | | | |

The modelling conducted in Studies 1 and 2 indicates that the reciprocal electrode arrangement provides equally as accurate bioimpedance estimates as the standard electrode arrangement. Given advantages offered by the reciprocal electrode arrangement with regards characterisation of the quality of electrode-to-tissue contact at the current electrodes and the ability to place the current electrodes very close to the region of interest, the reciprocal electrode arrangement can provide for significant advantages in the field of bioimpedance measurement.

### EXPERIMENTAL EXAMPLES

Testing was carried out on a healthy male subject, seated in a chair, with feet resting spaced apart on a rubber mat on the floor.

After allowing the subject to remain rested in a seated position for more than 5 minutes, electrodes were connected to the subject, generally in accordance with the configuration of electrodes shown in FIGS. 7a and 7b. In particular, four medium-sized oval-shaped adhesive gel electrodes (3.2 cm×5.7 cm) were attached in a linear configuration along the lateral surface of the left leg of the subject, proximal to the left malleolus. The spacing between the inner pair of the four electrodes (B,C) was 1 cm and the spacing between each one of the inner pair of electrodes (B,C) and its most adjacent outer electrode (A,D) was 2 cm. The electrodes were attached with oval long axis perpendicular to the length of the leg.

### Example 1

In Example 1, once the electrodes were in position, two sets of measurements were carried out. The first set of measurements was based on the standard electrode arrangement, as illustrated schematically in FIG. 7a, where an electrical current signal was applied between the outer pair of electrodes (A,D) and voltage measurements were made between the inner pair of electrodes (B,C). The second set of measurements was based on the reciprocal electrode arrangement, as illustrated schematically in FIG. 7b, where an electrical signal was applied between the inner pair of electrodes (B,C) and measurement of voltage between the outer pair of electrodes (A,D). The electrical signals were delivered at frequencies of 50 and 100 kHz, with constant current value of 250 μA.

Example 1 results are presented in Table 5, which shows the average resistance (R) and reactance (Xc) values determined for the region of interest (ROI) between the inner pair of electrodes (B,C). Each entry is the average of two determinations. Associated errors represent the measurement range.

TABLE 5

| | 50 kHz | | 100 kHz | |
|---|---|---|---|---|
| | R | Xc | R | Xc |
| Standard Z_{ROI} | 22.4 ± 0.2 | 4.9 ± 0.1 | 20.1 ± 0.2 | 4.2 ± 0.1 |
| Reciprocal Z_{ROI} | 22.3 ± 0.01 | 4.8 ± 0.05 | 19.9 ± 0.04 | 4.1 ± 0.1 |

As can be seen, bioimpedance impedance data (R & Xc) at the ROI is essentially equivalent for the standard and reciprocal electrode arrangements at applied frequencies 50 kHz and 100 kHz. Given advantages offered by the reciprocal electrode arrangement with regards characterisation of the quality of electrode-to-tissue contact at the current electrodes and the ability to place the current electrodes very close to the region of interest (e.g., a wound), the reciprocal electrode arrangement can provide for significant advantages in the field of bioimpedance measurement.

### Example 2

In Example 2, the same reciprocal electrode arrangement was used as for Example 1, except that a 100 ohm resistor was added alternately to each of the inner pair of electrodes (B,C) to simulate increased impedances associated with poor electrode-to-tissue contact at these electrodes. The electrical signals were delivered at frequencies of 100 kHz only, with constant current value of 250 μA. Resistance (R) and reactance (Xc) was determined based on voltage measurements between each possible pair combination of the electrodes (A-D, A-B, C-D, B-C).

Example 2 results are presented in Table 6. Table 6 shows that the addition of the resistor (in series) to either one of the inner pair of current electrodes (B,C) results in corresponding increases in resistances determined between that electrode and the adjacent one of the outer sensing voltage electrode (A,D). For example, the addition of the 100 ohm resistor in series to electrode B resulted in an increase of R(A-B), a measure of the transverse resistance under electrode B, of approximately 100 ohms. Likewise, the addition of 100 ohms resistor to electrode C resulted in an increase of R(C-D) of approximately 100 ohms. As was expected, there were no similar increases in reactance (Xc).

Thus, by establishing a suitable upper range of transverse resistance for the inner pair of current electrodes (B,C), in case of poor contact of the current electrodes, it is possible to use measurement of resistances R(A-B) and R(C-D) to indicate which of the current electrodes is at fault. It is important to identify poor quality contact so as to reduce the possibility of any further deterioration in the quality of the contact, which might prevent the device from delivering the set amount of current during measurement, and thus lead to erroneous impedance determination. A similar option for measuring the transverse impedance (and hence assessing contact status) is not available when using the standard electrode arrangement.

TABLE 6

| | 100 Ohm resistor position | | | | | | | |
|---|---|---|---|---|---|---|---|---|
| | R (A-D) | R (A-B) | R (C-D) | R (B-C) | Xc (A-D) | Xc (A-B) | Xc (C-D) | Xc (B-C) |
| No resistor | 20.9 | 42.0 | 50.1 | 112.7 | 3.7 | 28.1 | 32.5 | 64.8 |
| Electrode B | 20.9 | 141.7 | 50.0 | 213.3 | 3.8 | 28.6 | 32.8 | 62.5 |
| Electrode C | 20.9 | 42.5 | 149.2 | 211.9 | 3.7 | 26.8 | 28.2 | 63.1 |

Substantial consistency of the experimental data presented in Table 6 with the rule provided by Equation 5 (reproduced below and discussed further above) can also be clearly seen.

$$\frac{V ( B - C )}{I ( t )} = \frac{V ( A - D )}{I ( t )} + \frac{V ( A - B )}{I ( t )} + \frac{V ( C - D )}{I ( t )} \qquad [ 5 ]$$

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for characterization of body tissue, the method comprising:
applying a first electrical signal between an inner pair of current electrodes (B,C), the inner pair of current electrodes comprising a first current electrode (B) and a second current electrode (C) connected to tissue at a first side and a second side, respectively, of a tissue region of interest;
measuring one or more voltages between a first sensing electrode (A) and a second sensing electrode (D) of an outer pair of sensing electrodes (A,D) and/or between one of the outer pair of sensing electrodes (A,D) and one of the inner pair of current electrodes (B,C), the voltages resulting from the application of the first electrical signal, wherein the first sensing electrode (A) and the second sensing electrode (D) are connected to tissue at the first side and the second side, respectively, of the tissue region of interest, and at positions outside of the inner pair of electrodes (B,C); and
determining, from the one or more voltage measurements, bioimpedance (Z_{ROI}) across the tissue region of interest.

2. The method of claim 1, comprising measuring voltage (V(A-D)) between the outer pair of sensing electrodes (A,D) resulting from the application of the first electrical signal and determining bioimpedance (Z_{ROI}) across the tissue region of interest from the voltage measurement (V(A-D)).

3. The method of claim 1 or 2, comprising measuring voltage (V(A-B)) between the first electrodes (A,B) resulting from the application of the first electrical signal or a further electrical signal applied between the first and second current electrodes (B,C).

4. The method of claim 3, comprising determining, from the voltage (V(A-B)) measured between the first electrodes (A,B), bioimpedance (Z(A-B)) across tissue between the first electrodes (A,B).

5. The method of claim 3 or 4, comprising determining whether or not the voltage (V(A-B)) measured between the first electrodes (A,B) and/or the bioimpedance (Z(A-B)) determined across tissue between the first electrodes (A,B), is above or below a predetermined threshold level.

6. The method of any one of the preceding claims, comprising measuring voltage (V(C-D)) between the second electrodes (C,D)) resulting from the application of the first electrical signal or a further electrical signal applied between the first and second current electrodes (B,C).

7. The method of claim 6, comprising determining, from the voltage measurement (V(C-D)) between the second electrodes (C,D), bioimpedance (Z(C-D)) across tissue between the second electrodes (C,D).

8. The method of claim 6 or 7, comprising determining whether or not the voltage (V(C-D)) measured between the second electrodes (C,D) and/or the bioimpedance (Z(C-D)) determined across tissue between the second electrodes (C,D), is above or below a predetermined threshold level.

9. The method of claim 5 or 8, comprising, if the voltage or bioimpedance is above the predetermined level, issuing an alarm or other alert signal to indicate that poor electrode contact has been determined.

10. The method of any one of the preceding claims, comprising measuring voltage (V(A-C) or V(B-D)) between one of the outer pair of sensing electrodes (A,D) and the current electrode (B,C) that is on the opposite side of the region of interest from that sensing electrode, measuring voltage (V(C-D) or V(A-B)) between the other of the outer pair of sensing electrodes (A,D) and the current electrode (B,C) that is on the same side of the region of interest as that other sensing electrode, and determining bioimpedance (Z_{ROI}) across the tissue region of interest from these voltage measurements.

11. The method of any one of the preceding claims, comprising determining if the following equation is met:
$$\frac{V ( B - C )}{I ( t )} = \frac{V ( A - D )}{I ( t )} + \frac{V ( A - B )}{I ( t )} + \frac{V ( C - D )}{I ( t )} .$$

12. The methods of any one of the preceding claims, wherein the region of interest is a portion of the body that is a wound, a diseased muscle or a strained muscle.

13. Apparatus for characterization of body tissue, the apparatus comprising:
an inner pair of current electrodes (B,C), the inner pair of current electrodes comprising a first current electrode (B) and a second current electrode (C) adapted to connect to tissue at a first side and a second side, respectively, of a tissue region of interest;
a signal generator adapted to apply a first electrical signal between the inner pair of current electrodes;
an outer pair of sensing electrodes (A,D), the outer pair of electrodes comprising a first sensing electrode (A) and a second sensing electrode (D) adapted to be connected to tissue at the first side and the second side, respectively, of the tissue region of interest, and at positions outside of the inner pair of electrodes; and
a monitoring device adapted to measure one or more voltages between the first sensing electrode (A) and the second sensing electrode (D) of the outer pair of sensing electrodes (A,D) and/or between one of the outer pair of sensing electrodes (A,D) and one of the inner pair of current electrodes (B,C), resulting from the application of the first electrical signal, and determine, from the one or more voltage measurements, bioimpedance (Z_{ROI}) across the tissue region of interest.

14. The apparatus of claim 13, wherein the monitoring device is adapted to measure voltage (V(A-D)) between the outer pair of sensing electrodes (A,D) resulting from the application of the first electrical signal and determine bioimpedance (Z_{ROI}) across the tissue region of interest from the voltage measurement (V(A-D)).

15. The apparatus of claim 13 or 14, wherein the monitoring device is adapted to measure voltage (V(A-B)) between the first electrodes (A,B) resulting from the application of the first electrical signal or a further electrical signal applied between the first and second current electrodes (B,C).

16. The apparatus of claim 15, wherein the monitoring device is adapted to determine, from the voltage (V(A-B)) measured between the first electrodes (A,B), bioimpedance (Z(A-B)) across tissue between the first electrodes (A,B).

17. The apparatus of claim 15 or 16, wherein the monitoring device is adapted to determine whether or not the voltage (V(A-B)) measured between the first electrodes (A,B) and/or the bioimpedance (Z(A-B)) determined across tissue between the first electrodes (A,B), is above or below a predetermined threshold level.

18. The apparatus of any one of claims 13 to 17, wherein the monitoring device is adapted to measure voltage (V(C-D)) between the second electrodes (C,D)) resulting from the application of the first electrical signal or a further electrical signal applied between the first and second current electrodes (B,C).

19. The apparatus of claim 18, wherein the monitoring device is adapted to determine, from the voltage measurement (V(C-D)) between the second electrodes (C,D), bioimpedance (Z(C-D)) across tissue between the second electrodes (C,D).

20. The apparatus of claim 18 or 19, wherein the monitoring device is adapted to determine whether or not the voltage (V(C-D)) measured between the second electrodes (C,D) and/or the bioimpedance (Z(C-D)) determined across tissue between the second electrodes (C,D), is above or below a predetermined threshold level.

21. The apparatus of claim 17 or 20, wherein, if the voltage or bioimpedance is above the predetermined level, the apparatus is adapted to issue an alarm or other alert signal to indicate that poor electrode contact has been determined.

22. The apparatus of any one claims 13 to 21, wherein the monitoring device is adapted to measure voltage (V(A-C) or V(B-D)) between one of the outer pair of sensing electrodes (A,D) and the current electrode (B,C) that is on the opposite side of the region of interest from that sensing electrode, and measure voltage (V(C-D) or V(A-B)) between the other of the outer pair of sensing electrodes (A,D) and the current electrode (B,C) that is on the same side of the region of interest as that other sensing electrode, and determine bioimpedance (Z_{ROI}) across the tissue region of interest from these voltage measurements.

23. The apparatus of any one of claims 13 to 22, wherein the monitoring device is adapted to determine if the following equation is met:
$$\frac{V ( B - C )}{I ( t )} = \frac{V ( A - D )}{I ( t )} + \frac{V ( A - B )}{I ( t )} + \frac{V ( C - D )}{I ( t )} .$$

24. A method for characterization of body tissue, the method comprising:
applying a first electrical signal between a pair of current electrodes (B′, D′), the pair of current electrodes comprising a first current electrode (B′) and a second current electrode (D′), the first current electrode (B′) being connected to the surface of a tissue region of interest and the second current electrode (D′) being connected to tissue at a second side of the tissue region of interest;
measuring one or more voltages between a first sensing electrode (A′) and a sensing electrode (C′) and/or a third sensing electrode (E′), and/or between one of the first, second and third sensing electrodes (A′, C′, E′) and one of the pair of current electrodes (B′, D′), resulting from the application of the first electrical signal, wherein the first sensing electrode (A′) is connected to tissue at a first side of the tissue region of interest, substantially opposite to the second side, and the second and/or third sensing electrodes (C′, E′) are connected to tissue at the second side of the tissue region of interest; and
determining, from the one or more voltage measurements, bioimpedance (Z_{ROI}) across the tissue region of interest.

25. The method of claim 24, wherein the second sensing electrode (C′) is connected to tissue between the first and second current electrodes (B′, D′) and the third sensing electrode (E′) is connected to tissue on an opposite side of the second current electrode (D′) to the tissue region of interest.

26. The method of claim 24 or 25, comprising measuring voltage V(A′-C′) between the first and second sensing electrodes (A′, C′), during application of the first electrical signal, and determining bioimpedance (Z_{ROI}) across the tissue region of interest from the voltage measurement (V(A′-C′)).

27. The method of claim 25, 25 or 26, comprising measuring voltage (V(A′-B′)) between the first electrodes (A,B) resulting from the application of the first electrical signal or a further electrical signal applied between the first and second current electrodes (B′,D′).

28. The method of claim 27, comprising determining, from the voltage (V(A′-B′)) measured between the first electrodes (A′, B′), bioimpedance (Z(A′-B′)) across tissue between the first electrodes (A′, B′).

29. The method of claim 27 or 28, comprising determining whether or not the voltage (V(A′-B′)) measured between the first electrodes (A′, B′) and/or the bioimpedance (Z(A′-B′)) determined across tissue between the first electrodes (A′, B′), is above or below a predetermined threshold level.

30. The method of any one of claims 24 to 29, comprising measuring voltage (V(D′-E′)) between the second current electrode (D′) and the third sensing electrode (E′), resulting from the application of the first electrical signal or a further electrical signal applied between the pair of current electrodes (B′, D′).

31. The method of claim 30, comprising determining, from the voltage measurement (V(D′-E′)), bioimpedance (Z(D′-E′)) across tissue between the second current electrode (D′) and the third sensing electrode (E′).

32. The method of claim 30 or 31, comprising determining whether or not the voltage (V(D′-E′)) measured between the second current electrode (D′) and the third sensing electrode (E′) and/or the bioimpedance (Z(D′-E′)), determined across tissue between the second current electrode (D′) and the third sensing electrode (E′), is above or below a predetermined threshold level.

33. The method of claim 29 or 32, comprising, if the voltage or bioimpedance is above the predetermined level, issuing an alarm or other alert signal to indicate that poor electrode contact has been determined.

34. The method of any one of claims 24 to 33, comprising determining bioimpedance (Z_{ROI}) at the region of interest by measuring:
voltage (B′-C′) between the first current electrode (B′) and the second sensing electrode (C′) and voltage (V(A′-B′) between the first sensing electrode (A′) and the first current electrode (B′); and/or
voltage (A′-E′) between the first sensing electrode (A′) and the third sensing electrode (E′) and voltage (V(C′-E′) between the second sensing electrode (C′) and the third sensing electrode (E′); and/or
voltage (A′-D′) between the first sensing electrode (A′) and the second current electrode (D′) and voltage (V(C′-D′) between the second sensing electrode (C′) and the second current electrode (D′).

35. The method of any one of claims 24 to 34, comprising determining if the following equation is met:
$$\frac{V ( {B}^{′} - {D}^{′} )}{I ( t )} = \frac{V ( {A}^{′} - {C}^{′} )}{I ( t )} + \frac{V ( {A}^{′} - {B}^{′} )}{I ( t )} + \frac{V ( {D}^{′} - {E}^{′} )}{I ( t )} .$$

36. The method of any one of claims 24 to 35, wherein the region of interest is a portion of the body that is a wound, a diseased muscle or a strained muscle.

37. Apparatus for characterization of body tissue, the apparatus comprising:
a pair of current electrodes (B′, D′), the pair of current electrodes comprising a first current electrode (B′), adapted to connect to the surface of a tissue region of interest, and comprising a second current electrode (D′), adapted to connect to tissue at a second side of the tissue region of interest;
a signal generator adapted to apply a first electrical signal between the pair of current electrodes (B′, D′);
a plurality of sensing electrodes (A′, C′, E′), the sensing electrodes comprising a first sensing electrode (A′), adapted to connect to tissue at a first side of the tissue region of interest, substantially opposite to the second side, and comprising a second sensing electrode (C′) and/or a third sensing electrode (E′), adapted to connect to tissue at the second side of the tissue region of interest; and
a monitoring device adapted to measure one or more voltages between the first sensing electrode (A′) and the second and/or third sensing electrode (C′, E′) and/or between one of the sensing electrodes (A′, C′, E′) and one of the pair of current electrodes (B′, D′), resulting from the application of the first electrical signal, and determine, from the one or more voltage measurements, bioimpedance (Z_{ROI}) across the tissue region of interest.

38. The apparatus of claim 37, wherein the second sensing electrode (C′) is adapted to connect to tissue between the first and second current electrodes (B′, D′) and the third sensing electrode (E′) is adapted to connect to tissue on an opposite side of the second current electrode (D′) to the tissue region of interest.

39. The apparatus of 37 or 38, wherein the monitoring device is adapted to measure voltage V(A′-C′) between the first and second sensing electrodes (A′, C′), during application of the first electrical signal, and determine bioimpedance (Z_{ROI}) across the tissue region of interest from the voltage measurement (V(A′-C′)).

40. The apparatus of claim 37, 38 or 39, wherein the monitoring device is adapted to measure voltage (V(A′-B′)) between the first electrodes (A,B) resulting from the application of the first electrical signal or a further electrical signal applied between the first and second current electrodes (B′,D′).

41. The apparatus of claim 40, wherein the monitoring device is adapted to determine, from the voltage (V(A′-B′)) measured between the first electrodes (A′, B′), bioimpedance (Z(A′-B′)) across tissue between the first electrodes (A′, B′).

42. The apparatus of claim 40 or 41, wherein the monitoring device is adapted to determine whether or not the voltage (V(A′-B′)) measured between the first electrodes (A′, B′) and/or the bioimpedance (Z(A′-B′)) determined across tissue between the first electrodes (A′, B′), is above or below a predetermined threshold level.

43. The apparatus of any one of claims 37 to 42, wherein the monitoring device is adapted to measure voltage (V(D′-E′)) between the second current electrode (D′) and the third sensing electrode (E′), resulting from the application of the first electrical signal or a further electrical signal applied between the pair of current electrodes (B′, D′).

44. The apparatus of claim 43, wherein the monitoring device is adapted to determine, from the voltage measurement (V(D′-E′)), bioimpedance (Z(D′-E′)) across tissue between the second current electrode (D′) and the third sensing electrode (E′).

45. The apparatus of claim 43 or 44, wherein the monitoring device is adapted to determine whether or not the voltage (V(D′-E′)) measured between the second current electrode (D′) and the third sensing electrode (E′) and/or the bioimpedance (Z(D′-E′)), determined across tissue between the second current electrode (D′) and the third sensing electrode (E′), is above or below a predetermined threshold level.

46. The apparatus of claim 42 or 45, wherein, if the voltage or bioimpedance is above the predetermined level, the apparatus is adapted to issue an alarm or other alert signal to indicate that poor electrode contact has been determined.

47. The apparatus of any one of claims 37 to 47, wherein the monitoring device is adapted to determine bioimpedance (Z_{ROI}) at the region of interest by measuring:
voltage (B′-C′) between the first current electrode (B′) and the second sensing electrode (C′) and voltage (V(A′-B′) between the first sensing electrode (A′) and the first current electrode (B′); and/or
voltage (A′-E′) between the first sensing electrode (A′) and the third sensing electrode (E′) and voltage (V(C′-E′) between the second sensing electrode (C′) and the third sensing electrode (E′); and/or
voltage (A′-D′) between the first sensing electrode (A′) and the second current electrode (D′) and voltage (V(C′-D′) between the second sensing electrode (C′) and the second current electrode (D′).

48. The apparatus of any one of claims 37 to 47, wherein the monitoring device is adapted to determine if the following equation is met:
$$\frac{V ( {B}^{′} - {D}^{′} )}{I ( t )} = \frac{V ( {A}^{′} - {C}^{′} )}{I ( t )} + \frac{V ( {A}^{′} - {B}^{′} )}{I ( t )} + \frac{V ( {D}^{′} - {E}^{′} )}{I ( t )} .$$
